(19) United States
(12) Patent Application Publication
(10) Pub. No.: US 2026/0209670 A1
(43) Pub. Date: Jul. 23, 2026

(54) BIOLOGICAL REACTOR

(71) Applicant: GREENSKILL TECHNOLOGY HOLDINGS LTD, Edinburgh (GB)

(72) Inventor: Stephen Charles SKILL, St Austell Cornwall (GB)

(73) Assignee: GREENSKILL TECHNOLOGY HOLDINGS LTD, Edinburgh (GB)

(21) Appl. No.: 19/139,852

(22) PCT Filed: Dec. 21, 2023
(86) PCT No.: PCT/GB2023/053355

§ 371 Date: Jun. 16, 2025

(30) Foreign Application Priority Data
Dec. 22, 2022 (GB) 2219552.3

(51) Int. Cl. C12M 1/00; C12M 1/12; C12M 1/34; C12M 3/04

(52) U.S. Cl. CPC C12M 27/22; C12M 21/02; C12M 23/06; C12M 27/10; C12M 29/18; C12M 41/06; C12M 41/12

(57) ABSTRACT

A bioreactor (1) comprises: a housing (2) defining an internal volume configured to hold a body of fluid; at least one inlet (23a, 23b) for ingress of fluid into the internal volume; at least one outlet (24a, 24b) for egress of fluid from the internal volume; a rotor assembly (3) comprising a rotatably mounted rotor shaft (30); a plurality of fluid contact elements (5) rotatable with the rotor shaft (30); wherein at least one fluid contact element (5) comprises a scoop (54) having a curved radially inner wall (53), the curved radially inner wall being concave when viewed from a central axis of the housing; wherein adjacent fluid contact elements (5) are spaced apart in a circumferential direction.

This invention relates generally to a bioreactor and a method of operation thereof. More specifically, although not exclusively, this invention relates to a bioreactor in the field of bioengineering, and in particular to a horizontally configured bioreactor for the cultivation of cells and/or tissues.

With the continuous development of the biotechnology industry, there is an urgent need to provide bioreactors for large-scale cultivation of microorganisms, animals, and plant cells. In the past, mechanically stirred reactors (also called reaction tanks) with improved chemical industry production equipment have been used. Due to the large shearing force of this type of mechanically stirred reaction tank during the stirring of the mixture, when used in animal tissue, plant tissue or fragile microorganism cell culture, cells are often not protected by a cell wall. As a result, they cannot withstand vigorous stirring and aeration, and are easily damaged.

Furthermore, it has been observed that the bubbles formed when the stirrer rotates at high speed will also damage sensitive cells. In order to reduce the shearing force of the stirrer blades on the cells and enhance the mass transfer capacity of the reactor, many improvements have been made to the traditional mechanical stirring method and gas, heat and mass transfer methods of the reactor.

Due to the variety of cell survival conditions, prior devices have not fundamentally solved the problems of damage to cells due to large mixing and shearing forces, uneven material transfer, internal material accumulation, large head spaces and dead corners in equipment in large-scale cultivation. The present invention is intended to overcome one or more of these drawbacks.

The present invention provides a bioreactor with unique stirring performance, low mechanical stirring shear force, little interference of cell growth process on cell growth, high gas mass transfer, high-density stepwise or continuous flow cell culture, and/or online product collection. The present invention may also be used for cell suspension culture or microcarrier culture of adherent cells.

One purpose of the present invention may be a device for high-density in vitro cultivation of animal cells, plant cells, algae, fungi, bacteria, viruses or consortia thereof, and it is collectively referred to as “bioreactor” in this document.

It is therefore a first non-exclusive object of the invention to provide an improved bioreactor.

Accordingly, an aspect of the invention provides bioreactor comprising: a housing defining an internal volume configured to hold a body of fluid; at least one inlet for ingress of fluid into the internal volume; at least one outlet for egress of fluid from the internal volume; a rotor assembly comprising a rotatably mounted rotor shaft; a plurality of fluid contact elements rotatable with the rotor shaft; wherein at least one fluid contact element comprises a scoop having a curved radially inner wall, the curved radially inner wall being concave when viewed from a central axis of the housing; wherein adjacent fluid contact elements are spaced apart in a circumferential direction.

The fluid may comprise a liquid, gas or a combination of liquid and gas. The fluid may further comprise a solid entrained in a liquid, gas or a combination of liquid and gas.

The fluid contact elements may be or comprise fluid, liquid and/or gas contact elements, fluid, liquid and/or gas agitation elements or liquid, solid and/or gas contact elements.

The bioreactor may comprise at least two inlets. Additionally or alternatively, the bioreactor may comprise at least two outlets.

Each outlet may be configured to remove or extract a different phase of material located within the bioreactor, e.g. in use.

The radially inner wall may comprise one or more apertures. The radially inner wall may comprise a plurality of apertures. The radially inner wall may comprise one or more perforations. The radially inner wall may comprise a plurality of perforations.

The radially outer wall may comprise one or more apertures. The radially outer wall may comprise a plurality of apertures. The radially outer wall may comprise one or more perforations. The radially outer wall may comprise a plurality of perforations.

The radially inner wall may comprise a lip portion. The one or more apertures may be formed in the lip portion.

The at least one fluid contact element may further comprise a radially outer wall, side walls extending between the radially inner and outer walls, and an opening between the radially inner and outer walls at one end of the side walls.

At least one fluid contact element may further comprise a first end wall extending between the radially inner and outer walls at the other end of the side walls.

The opening may define or provide an inlet to the fluid contact element.

The opening or inlet may be defined by the radially inner wall and outer wall and side walls One or each of the side walls may comprise one or more apertures or perforations.

The radially inner wall may comprise one or more apertures adjacent to the opening between the radially inner and outer walls.

The radially inner wall may comprise one or more apertures or perforations adjacent or proximate to the opening between the radially inner and outer walls.

One or each of the side walls may comprise one or more apertures of perforations adjacent or proximate to the opening between the radially inner and outer walls.

The side walls together with the radially inner and outer walls and first end wall may describe a receptacle for holding fluid.

In some examples, the fluid element comprises a scoop or bucket. The fluid contact element may comprise a trough. The fluid contact element may define a receptacle for fluid.

The radially inner and outer walls and first end wall may be integrally formed.

The radially inner and outer walls may be interconnected by the first end wall.

The radially inner wall may comprise a front retaining portion extending from a first end thereof towards the radially outer wall and configured to retain fluid within the fluid contact element, in use. The opening may be defined between the radially outer wall and front retaining portion.

The front retaining portion may comprise a lip portion extending therefrom towards the first end wall of the fluid contact element. The one or more apertures or perforations may be formed in or through the lip portion. The opening or inlet may be defined between the radially outer wall and front retaining portion.

The fluid contact element may comprise a partition wall disposed between the side walls.

The partition wall may separate the fluid contact element into separate volumes.

One or more, or each fluid contact element may be individually removably mounted within the housing.

The rotor shaft may extend from an exterior of the housing into the internal volume.

The bioreactor may comprise a concentric scoop assembly. The concentric scoop assembly may define or comprise the plurality of fluid contact elements. The concentric scoop assembly may be configured to be received within the housing.

The concentric scoop assembly may comprise concentric inner and outer tubes. The inner and outer tubes may define an annulus. The fluid contact elements may be defined or provided between the inner and outer tubes. The inner tube may define the radially inner wall. The outer tube may define the radially outer wall.

The concentric scoop assembly may be rotatable with the rotor shaft.

The concentric scoop assembly may comprise a plurality of annular walls provided within the annulus and spaced, e.g. equally, along the longitudinal axis. The annular walls may connect the inner and outer tubes and may define side walls of the fluid contact elements.

The concentric scoop assembly may comprise a plurality of longitudinally extending walls spaced, e.g. equally, around the annulus. The longitudinally extending end walls may connect the inner and outer tubes and may define end walls of the fluid contact elements.

The inner tube may comprise a plurality of perforations or apertures.

The outer tube may comprise a plurality of perforations or apertures. The perforations in the outer tube may be configured to provide fluid communication between one or more of the plurality of fluid contact elements and a region between the concentric scoop assembly and an inner wall of the housing.

The rotor shaft may extend along the length of the housing within the internal volume. The rotor shaft may comprise a pair of stub shafts, each respective stub shaft connected with a respective end wall of the housing. The stub shafts may not extend into the internal volume.

The rotor assembly may comprise a driving means configured to rotate the rotor shaft. The driving means may comprise a motor. The driving means may comprise a magnetic drive.

The bioreactor may comprise at least one plate member mounted on, and configured to rotate with, the rotor shaft within the internal volume.

The bioreactor may comprise a plurality of spaced apart plate members mounted on the rotor shaft. The bioreactor may comprise a plurality of fluid contact elements extending between adjacent plate members.

At least one fluid contact element may extend between adjacent plate members. At least one fluid contact element may extend between a plate member and an end wall of the housing.

In some examples, the side walls of at least one fluid contact element may be provided by or defined by adjacent plate members.

A plurality of fluid contact elements may extend between adjacent plate members.

At least one fluid contact element may be located proximate to the periphery of the adjacent plate members. At least one fluid contact element may be configured to rotate with the or adjacent plate members, in use.

The bioreactor may comprise a plurality of fluid contact elements circumferentially spaced around adjacent plate members.

The bioreactor may be a photobioreactor. The photobioreactor may comprise a source of electromagnetic radiation configured to transmit electromagnetic radiation to the internal volume.

The source of electromagnetic radiation may be located externally of the housing.

The bioreactor may comprise transmission means for transmitting the electromagnetic radiation to the at least one plate member.

The plate member may be configured to direct the electromagnetic radiation from a surface of the at least one plate member into the internal volume.

The transmission means may be rotatable with the rotor shaft.

The rotor shaft may comprise the transmission means.

The at least one plate member may comprise a planar surface and may be arranged to project electromagnetic radiation from the planar surface into the internal volume, in use.

The at least one plate member may be arranged to project electromagnetic radiation from an edge portion or edge surface into the internal volume, in use.

The transmission means may comprise a beam deflector. The or a beam deflector may be arranged to direct electromagnetic radiation to the at least one plate member, in use.

The or a beam deflector may be arranged to direct electromagnetic radiation from the rotor shaft to the at least one plate member, in use.

The beam deflector may comprise a prism. The beam deflector may comprise a surface etching and/or surface coating. The beam deflector may comprise an insert. The beam deflector may comprise a mirror coating or reflective coating.

The at least one plate member may be transmissive to the electromagnetic radiation. In some examples, the rotor shaft may be transmissible to electromagnetic radiation.

The at least one plate member may comprise at least one beam deflector which deflects electromagnetic radiation out of the plate member into the internal volume, in use.

The source of electromagnetic radiation may comprise one or more light emitting diodes (LEDs).

The source of electromagnetic radiation may comprise one or more laser diodes.

The source of electromagnetic radiation may comprise one or more optical fibres transmitting electromagnetic radiation (i.e. captured and transmitted sunlight).

The rotor shaft and/or at least one plate member may be formed of a substantially translucent or transparent material. The rotor shaft and/or at least one plate member may be transmissible to electromagnetic radiation.

The source of electromagnetic radiation may be configured to transmit electromagnetic radiation into the substantially translucent or transparent material of the rotor shaft.

In some examples, the means for projecting the electromagnetic radiation comprises at least one source of electromagnetic radiation mounted on the at least one plate member.

The means for projecting the electromagnetic radiation may comprise at least one source of electromagnetic radiation mounted on a planar surface of the at least one plate member.

Additionally or alternatively, the at least one source of electromagnetic radiation may be mounted on an edge portion or edge surface of the at least one plate member. The at least one source of electromagnetic radiation may extend across a planar surface of the at least one plate member.

The bioreactor may comprise a plurality of sources of electromagnetic radiation mounted on the at least one plate member.

The plurality of sources of electromagnetic radiation may be arranged in an array.

At least one source of electromagnetic radiation may comprise a light emitting diode, a laser diode or fibre optic source.

The at least one source of electromagnetic radiation may comprise an array of light emitting diodes, laser diodes or fibre optic sources.

The bioreactor may comprise a source of electricity. The source of electricity may be located externally of the housing. The photobioreactor may comprise one or more conductors connecting the source of electricity to the at least one source of electromagnetic radiation.

The one or more conductors may be configured to rotate with the rotor shaft.

The bioreactor may comprise commutator means for connecting the source of electricity to the one or more conductors.

The rotor shaft and at least one plate member may submersible in the body of fluid, e.g. in use.

The bioreactor may be arranged such that, in use, the housing is arranged with its longitudinal axis substantially horizontally disposed. The bioreactor may be arranged such that, in use, the rotor shaft is arranged with its longitudinal axis substantially horizontally disposed.

A major portion of the housing may be opaque to the electromagnetic radiation.

The housing may comprise a substantially tubular wall or substantially tubular side wall and two end walls. The rotor shaft may extend between the end walls.

The at least one plate member may partition the internal volume into a plurality of sub-volumes. The sub-volumes may be fluidly connected volumes.

The bioreactor may comprise one or more heating and/or cooling elements located within the internal volume.

The bioreactor may comprise one or more heating and/or cooling elements located externally of or outside of the internal volume.

According to another aspect of the invention, there is provided a bioreactor comprising: a housing defining an internal volume configured to hold a body of fluid; at least one inlet for ingress of fluid into the internal volume; at least one outlet for egress of fluid from the internal volume; a rotor assembly comprising a rotatably mounted rotor shaft; at least one fluid contact element rotatable with the rotor shaft; wherein the at least one fluid contact element comprises a scoop having a wall with one or more apertures extending therethrough.

According to another aspect of the invention, there is provided a bioreactor comprising: a housing having an internal volume configured to hold a body of fluid; at least one inlet for ingress of fluid into the internal volume; at least one outlet for egress of fluid from the internal volume; a rotor assembly comprising a rotatably mounted rotor shaft; at least one fluid contact element rotatable with the rotor shaft; wherein the at least one fluid contact element comprises a scoop.

Accordingly, an aspect of the invention provides a bioreactor comprising: a housing defining an internal volume configured to hold a body of fluid; at least one inlet for ingress of fluid into the internal volume; at least one outlet for egress of fluid from the internal volume; a rotor assembly comprising a rotatably mounted rotor shaft extending from an exterior of the housing into the internal volume and at least one plate member mounted on, and configured to rotate with, the rotor shaft within the internal volume; and means for projecting electromagnetic radiation from the at least one plate member into the internal volume.

According to another aspect of the invention, there is provided a bioreactor comprising: a housing defining an internal volume configured to hold a body of fluid; at least one inlet for ingress of fluid into the internal volume; at least one outlet for egress of fluid from the internal volume; a rotor assembly comprising a rotatably mounted rotor shaft extending from an exterior of the housing into the internal volume and at least one plate member mounted on, and configured to rotate with, the rotor shaft within the internal volume; a source of electromagnetic radiation located externally of the housing; and transmission means for transmitting the electromagnetic radiation to the at least one plate member, wherein the plate member is configured to direct the electromagnetic radiation from a surface of the at least one plate member into the internal volume.

According to another aspect of the invention, there is provided a bioreactor comprising: a housing defining an internal volume configured to hold a body of fluid; at least one inlet for ingress of fluid into the internal volume; at least one outlet for egress of fluid from the internal volume; a rotor assembly comprising a rotatably mounted rotor shaft extending from an exterior of the housing into the internal volume and at least one plate member mounted on, and configured to rotate with, the rotor shaft within the internal volume; and at least one source of electromagnetic radiation mounted on the at least one plate member and configured to project the electromagnetic radiation into the internal volume.

According to another aspect of the invention, there is provided a bioreactor comprising: a housing defining an internal volume configured to hold a body of fluid; at least one inlet for ingress of fluid into the internal volume; at least one outlet for egress of fluid from the internal volume; a rotor assembly comprising a rotatably mounted rotor shaft extending from an exterior of the housing into the internal volume and at least one plate member mounted on, and configured to rotate with, the rotor shaft within the internal volume; and at least one fluid contact element mounted on, and rotatable with, the at least one plate member, wherein the at least one fluid contact element comprises radially inner and outer walls, side walls extending between the radially inner and outer walls; a first end wall extending between the radially inner and outer walls at one end of the side walls and an opening between the radially inner and outer walls at the other end of the side walls, wherein the radially inner wall comprises one or more apertures extending therethrough.

In some examples, the bioreactor is configured to be operated in a dark fermentation process.

In some examples, the bioreactor is configured to be operated in an extractive fermentation process.

In some examples, the bioreactor is configured such that the internal volume can be operated at a pressure of between 0 and 1000 bar (between 0 and 100000 kPa).

In some examples, the bioreactor is configured such that the internal volume can be operated under vacuum.

According to another aspect of the invention, there is provided a method of operating a bioreactor described above, wherein the bioreactor is more than 50% full of fluid, the method comprising rotating the rotor shaft such that at least one fluid contact element passes through the fluid.

In some examples, the bioreactor is more than 50% full of liquid.

In some examples, the fluid comprises two or more liquids having different densities, and the at least one fluid contact element passes through each of the two liquids.

In some examples, the fluid comprises two or more liquids having different densities containing entrained solids and the at least one fluid contact element passes through each of the two or more liquids containing entrained solids.

In some examples, the fluid comprises a gas and a liquid, and the at least one fluid contact element passes through each of the gas and liquid.

In some examples, the fluid comprises a gas and two or more liquids, and the at least one fluid contact element passes through each of the gas and two liquids.

For example, there may be a lower dense phase of perfluorocarbon solvent, overlain by an aqueous phase, overlain by an organic solvent phase (dodecane), overlain by a gas phase.

In some examples, the fluid comprises a gas and two or more liquids having different densities. There may be entrained solids within at least one of the liquids.

In some examples, the solids can be adsorbent particles such as ion exchange matrices or affinity sorbents for the recovery of specific molecules (e.g. antibodies). The solids may be designed to either float or sink or a combination of both.

In some examples, the at least one fluid contact element passes through each of the gas and two or more liquids with entrained solids.

In some examples, the method comprises operating the bioreactor in a dark fermentation process.

In some examples, the method comprises operating the bioreactor housing and fluid contents at a pressure of between 0 and 1000 bar (between 0 and 100000 kPa).

In some examples, the method comprises operating the bioreactor housing and fluid contents under vacuum.

For the avoidance of doubt, any of the features described herein apply equally to any aspect of the invention.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example only with reference to the accompanying drawings in which:

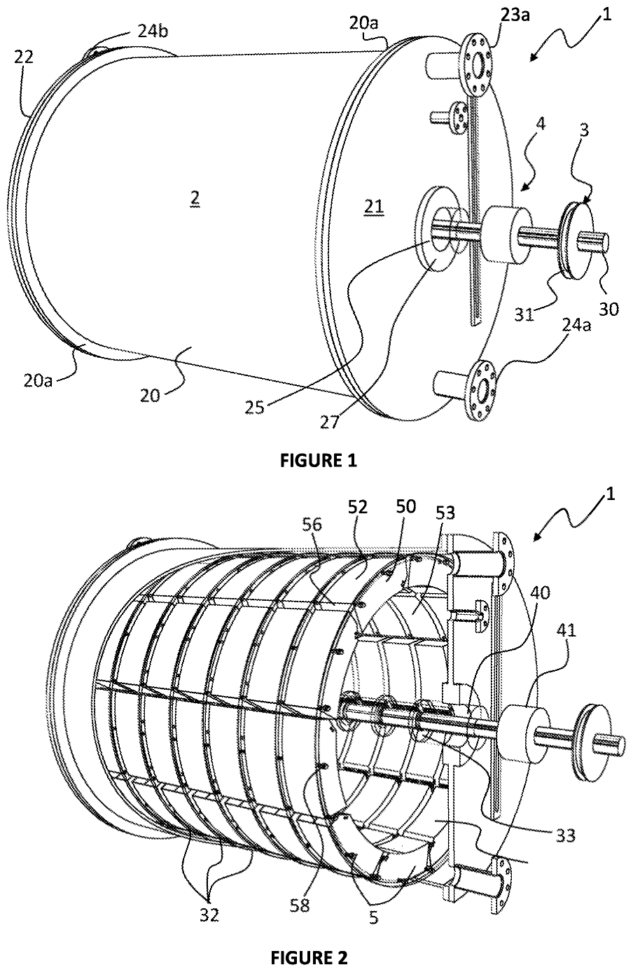

FIG. 1 is a perspective view of a photobioreactor according to an embodiment of the invention;

FIG. 2 is a perspective view of the photobioreactor of FIG. 1 with the side wall and first end wall removed;

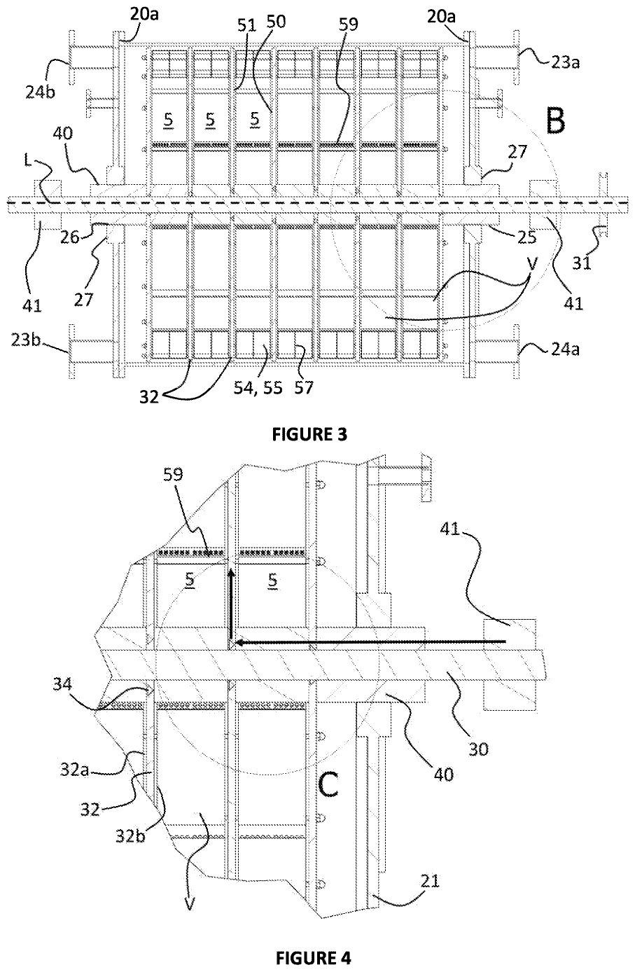

FIG. 3 is a side sectional view of the photobioreactor of FIG. 1;

FIG. 4 is a detail view of region B of FIG. 3;

FIG. 5 is a detail view of region C of FIG. 4;

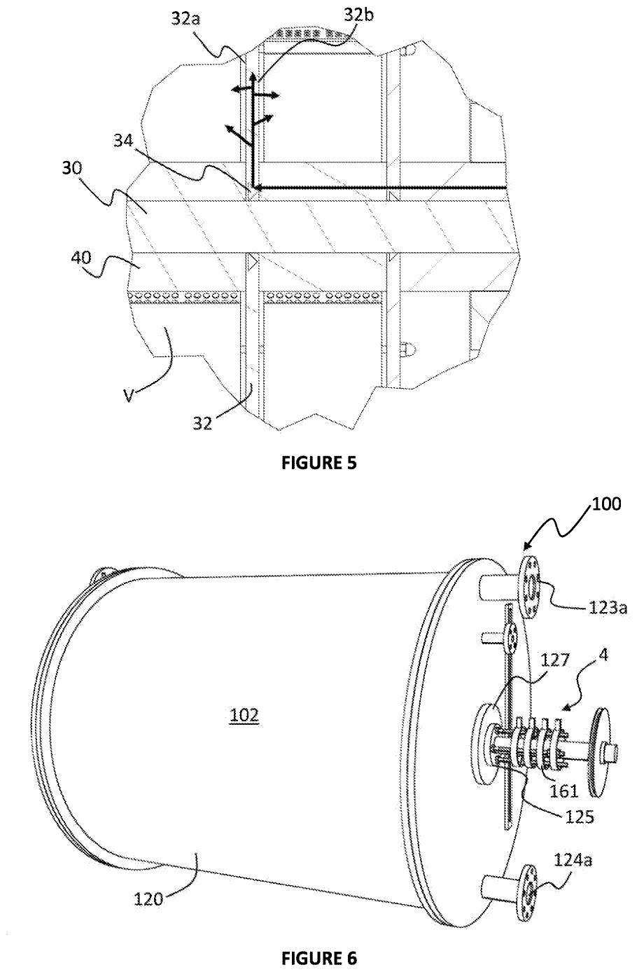

FIG. 6 is a perspective view of a photobioreactor according to another embodiment of the invention;

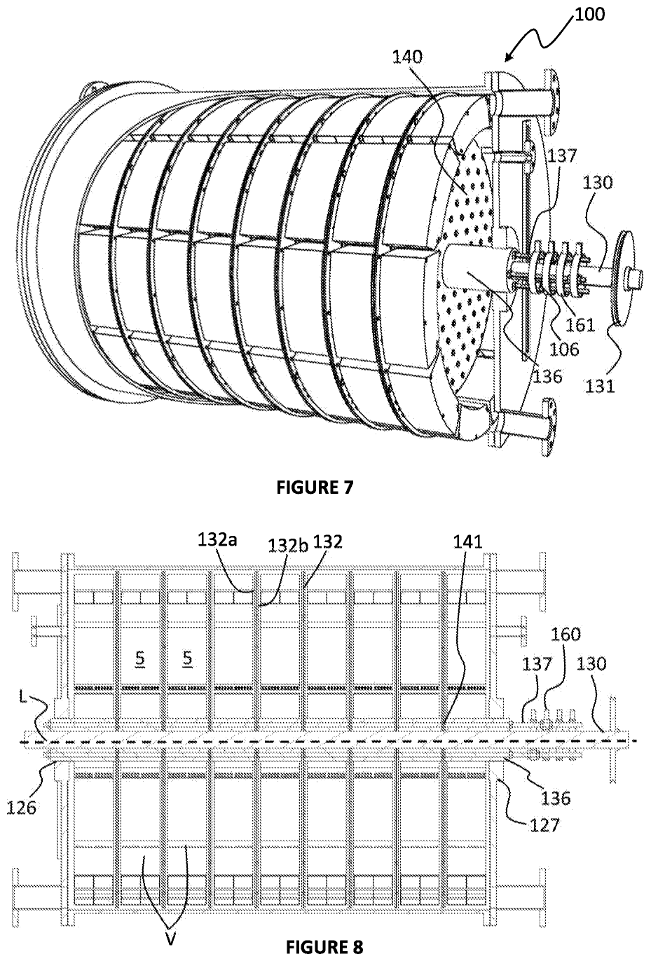

FIG. 7 is a perspective view of the photobioreactor of FIG. 6 with the side wall and first end wall removed;

FIG. 8 is a side sectional view of the photobioreactor of FIG. 6;

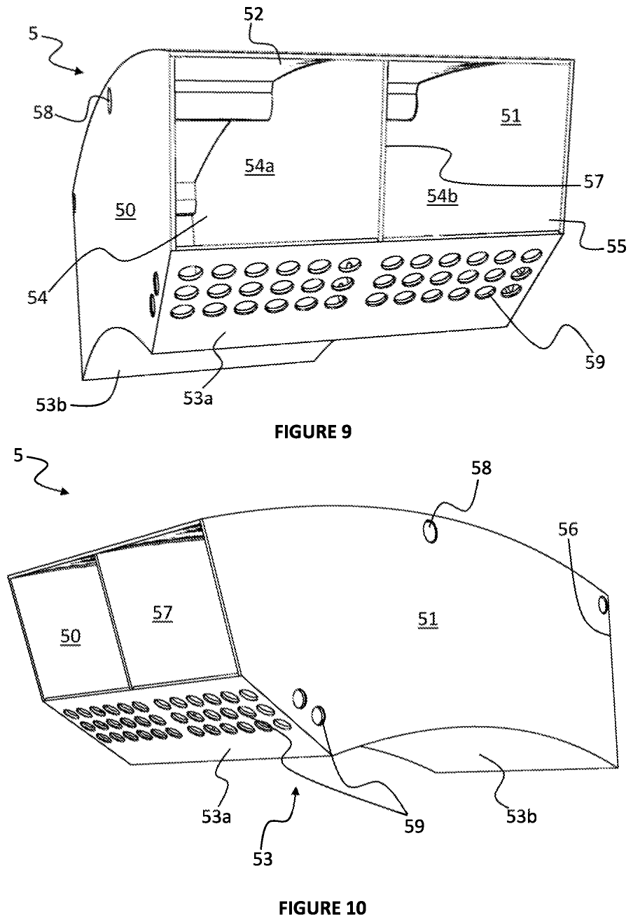

FIG. 9 is a front perspective view of a fluid contact element according to an embodiment of the invention;

FIG. 10 is a rear perspective view of the fluid contact element of FIG. 9;

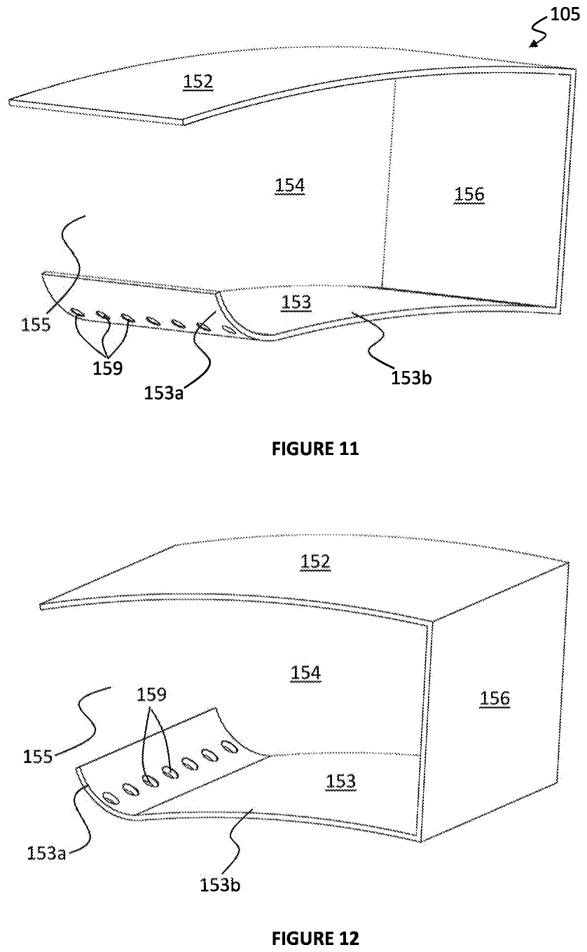

FIG. 11 is a front perspective view of a fluid contact element according to an embodiment of the invention;

FIG. 12 is a rear perspective view of the fluid contact element of FIG. 11;

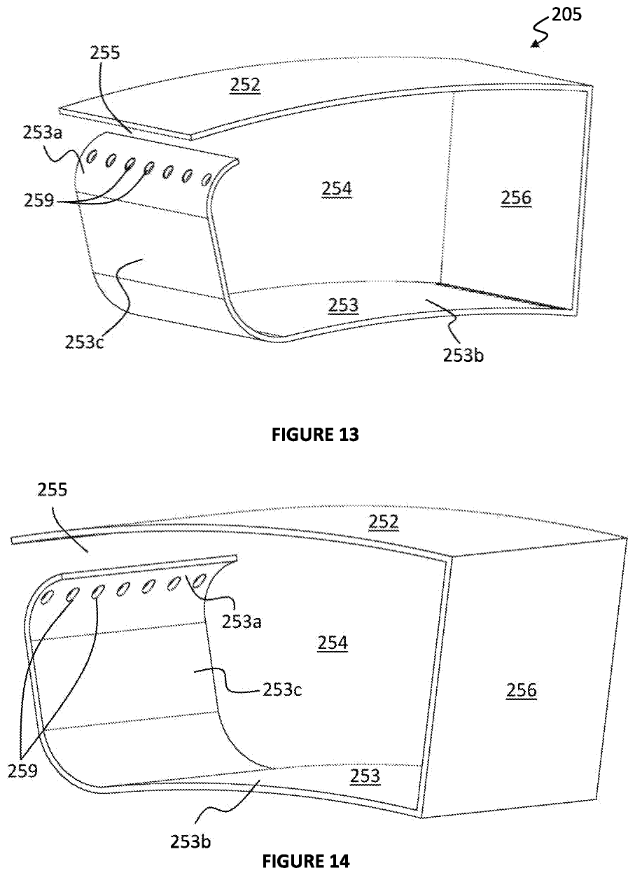

FIG. 13 is a front perspective view of a fluid contact element according to an embodiment of the invention;

FIG. 14 is a further perspective view of the fluid contact element of FIG. 13;

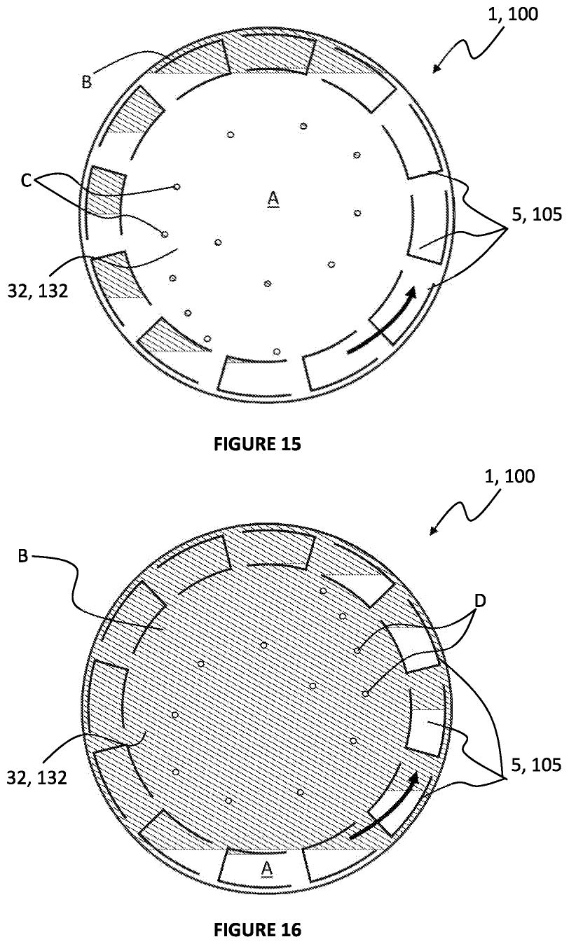

FIG. 15 is a cross-sectional view of a photobioreactor according to an embodiment of the invention containing first fluid phase arrangement;

FIG. 16 is a cross-sectional view of a photobioreactor according to an embodiment of the invention containing second fluid phase arrangement;

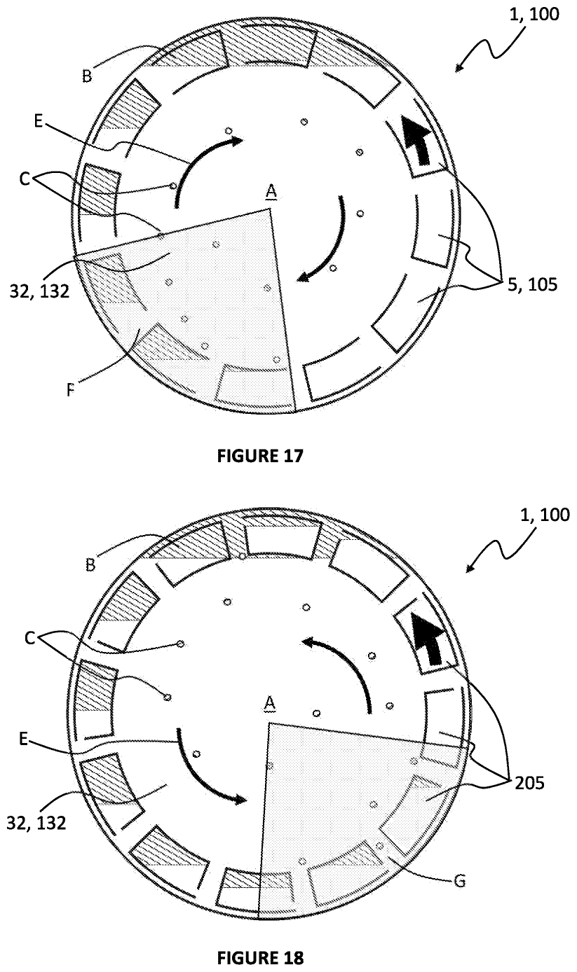

FIG. 17 is a cross-sectional view of a photobioreactor according to an embodiment of the invention showing gas bubble release from a first fluid contact element design within the fluid phase arrangement of FIG. 15;

FIG. 18 is a cross-sectional view of a photobioreactor according to an embodiment of the invention showing gas bubble release from a second fluid contact element design within the fluid phase arrangement of FIG. 15; and

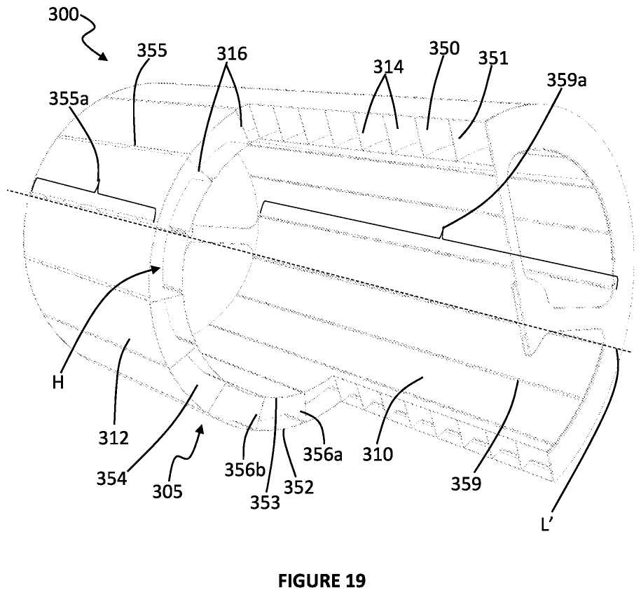

FIG. 19 is a perspective partial cutaway view of a concentric scoop assembly of a photobioreactor according to another embodiment of the invention.

Referring now to FIG. 1, there is shown a photobioreactor 1 according to an embodiment of the invention. The photobioreactor 1 includes a tubular housing 2 in the form of a cylinder of circular cross-section that defines an internal volume arranged to receive and hold a body of fluid. The internal volume may alternatively be defined as a reactor vessel or contact chamber. The internal volume is configured to receive a fluid to be treated, in the form of a waste liquid stream in this example, and is configured to hold a culture medium arranged to contact and treat the waste liquid stream. The photobioreactor 1 further includes a rotor assembly 3 extending into, and through, the tubular housing 2 and configured to impart rotation to a plurality of fluid contact elements 5 (shown in greater detail in FIGS. 9 and 10) so to agitate fluid within the housing 2 and provide greater contact therebetween. A lighting unit 4 is arranged to transmit light into the internal volume from a location external thereof.

Referring to FIGS. 1 to 3, the tubular housing 2 includes a sidewall 20 of circular cross-section connected with and extending between a pair of opposed circular end walls 21, 22 and defines the internal volume. The tubular housing 2 is arranged such that its longitudinal axis L (as shown in FIG. 3) is disposed substantially horizontally, in use.

The side wall 20 has a radial flange 20a at either end in order to facilitate connection with the end walls 21, 22. Each of the side wall 20 and end walls 21, 22 are opaque in this example and may be formed of a metallic and/or polymeric material. A first inlet 23a extends through a first of the end walls 21 and a second inlet 23b (as shown in FIG. 3) extends through a second of the end walls 22. In use, with the housing 2 substantially horizontally disposed, the first inlet 23a is located higher than the second inlet 23b. Each inlet 23a, 23b is configured to feed fluid into the internal volume, in which the first inlet 23a is configured to feed a waste liquid stream to be treated and the second inlet 23b is configured to feed a culture medium. A first outlet 24a extends through a first of the end walls 21 and a second outlet 24b extends through a second of the end walls 22, wherein each outlet is configured to extract fluid from the internal volume. In use, with the housing 2 horizontally disposed, the first outlet 24a is located lower than the second outlet 24b.

Each of the end walls 21, 22 includes a respective central aperture 25, 26 for receipt of the rotor assembly 3 and one or more components of the lighting unit 4 (described in greater detail below). An annular seal 27 is provided between each of the central apertures 25, 26 and rotor assembly 3/lighting unit 4, in order to prevent the leakage of fluid from the internal volume.

The rotor assembly 3 includes a rotor shaft 30 extending through the central aperture 25, along the tubular housing 2 and through the central aperture 26. A pulley wheel 31 is connected to, and rotatable with the rotor shaft 30. The pulley wheel 31 is configured to be connected to a pulley belt and motor (not shown) in order to impart rotation to the rotor shaft 30, in use.

As shown in greater detail in FIGS. 2 and 3, the rotor shaft 30 carries a plurality of radially extending plate members 32 that are spaced from one another and are configured to rotate with the rotor shaft 30, in use. The plate members 32 are light-transmissible and formed of Poly(methyl methacrylate) (PMMA) in this example. The plate members 32 also have light diffusion particles disposed throughout in order to allow for a more even emission of light, in use.

Each of the plate members 32 includes a central aperture 33 to allow for passage of the rotor shaft 30. As shown more clearly in FIG. 3, the plate members 32 partition the internal volume into respective fluidly connected volumes V. Each volume V is bounded by adjacent plate members 32 and the side wall 20 of the housing 2.

The lighting unit 4 is configured to transmit light to the internal volume of the photobioreactor 1 and includes a light column 40 having a plurality of individual tubular segments in this example. The light column 40 surrounds the rotor shaft 30 and extends along the length thereof and through each of the central apertures 25, 26. The annular seals 27, described above, seal between each of the apertures 25, 26 and the light column 40. The light column 40 is light-transmissible and formed of Poly(methyl methacrylate) (PMMA) in this example. The lighting unit 4 also includes a pair of opposed light sources 41 each mounted to the rotor shaft 30 external of the internal volume and spaced from respective ends of the light column 40. Each of the light sources 41 include a plurality of LEDs and are operable to transmit light into and along the light column 40, in use.

As shown in greater detail in FIGS. 4 and 5, each radially extending plate member 32 includes a beam deflector 34 located proximate the central aperture 33 and extending therearound. The beam deflector 34 is a 45 degree reflecting surface machined into the material of the plate member 32 and is configured to direct light from the light column 40 along a respective plate member 32 (as shown in FIG. 4). The plate members 32 are configured to emit the transmitted light from their respective planar surfaces 32a, 32b in order to illuminate the internal volume, as shown in FIG. 5. In use, each respective volume V receives transmitted light from the planar surfaces of adjacent plate members 32.

As shown in FIGS. 2 and 3, each of the radially extending plate members 32 carries a plurality of circumferentially spaced fluid contact elements 5. The fluid contact elements 5 are described in greater detail below in relation to FIGS. 9 and 10, but a brief description is provided here in the context of the photobioreactor 1. The fluid contact elements 5 are mounted between adjacent plate members 32 and are located proximate the outer periphery of the plate members 32. The fluid contact elements 5 are provided as scoops/buckets in this example and are arranged to rotate with the plate members 32, in use.

The fluid contact elements 5 have a pair of side walls 50, 51 connecting a curved radially inner wall 53 and radially outer wall 52 that define opposed bounding walls. The side walls 50, 51 and bounding walls 52, 53 define a scoop/bucket portion 54 for receipt of fluid during rotation of the plate members 32. An opening or inlet is defined at a first end 55 and a first end wall 56 is provided at a second end. A partition wall 57 is provided between the side walls 50, 51 so as to separate the scoop portion 54 into separate volumes. The side walls 50, 51 are provided with apertures 58 configured to receive means for fixing the fluid contact elements 5 between adjacent plate members 32. The plate members 32 also have corresponding apertures (not shown) for receipt of said fixing means. The radially inner wall 53 has a plurality of perforations 59 extending therethrough to allow fluid communication between the scoop portion 54 and the internal volume.

In use, a liquid on which a fermentation, treatment or cultivation process is to be carried out is fed into the internal volume through the first inlet 23a. A culture medium is fed into the internal volume through the second inlet 23b. The culture medium is phototrophic in this example, relying upon light to efficiently carry out the required biochemical processes.

One or each of the pair of light sources 41 is activated such that light is transmitted from a location external of the internal volume and along the light column 40. The light passing along the light column 40 is deflected by the beam deflectors 34 and transmitted along each of the plate members 32. The diffusion particles disposed throughout the plate members 32 allow the transmitted light to be emitted evenly from each of the planar surfaces 32a, 32b and into the internal volume.

Further, whilst the light is being transmitted into the internal volume, the rotor assembly 3 rotates the rotor shaft 30, and, in turn, each of the plate members 32 and fluid contact elements 5. Due to the horizontal disposition of the housing 2 during use, the fluid contact elements 5 collect liquid from the bottom of the internal volume and transport it around the circumference of the housing 2 as they are rotated. The fluid is retained it within the scoop portion 54. However, at a given orientation of the fluid contact elements 5 during rotation, the perforations 59 create a rain effect due to releasing a portion of the liquid retained within the scoop portion 54 (as will be described in more detail with reference to FIGS. 15 and 16). The rain effect increases the surface area of the liquid exposed to the light and also encourages mixing of the waste liquid stream and the culture medium. The rain effect also provides a cleaning effect to the plate members 32, helping to reduce the build-up of algae and therefore maintain the light emissive properties thereof.

It will be appreciated by those skilled in the art that several variations to the aforementioned embodiments are envisaged without departing from the scope of the invention. For example, the light column is described has having a plurality of segments. However, this need not be the case, instead, the light column may be provided as a single, unitary shaft or column. Further, in some examples, the rotor shaft may be transparent thereby negating the need for a light column as described.

Whilst the fluid contact elements have been described as having a particular arrangement, this need not be the case. The fluid contact elements of any of FIGS. 11 to 14 or FIG. 19, described below, may be incorporated into the aforementioned photobioreactor.

Further, whilst the housing has been described as cylindrical, this need not be the case. The housing may have any other suitable shape capable of providing an internal volume to house the rotor assembly and fluid contact elements as described.

Whilst the plate members and light column have been described as being formed of PMMA, this need not be the case. Instead, the plate members and/or light column may be formed of any other suitable polymeric material or may be formed of glass.

Referring now to FIGS. 6 to 8, there is shown a photobioreactor 100 according to another example. The photobioreactor 100 is similar to the photobioreactor 1, and like features will be denoted by like reference numerals incremented by ‘100’. Photobioreactor 100 differs from photobioreactor 1 in the arrangement of the rotor assembly 103 and the lighting unit 104 as described in greater detail below.

In a similar manner to photobioreactor 1, the rotor assembly 103 includes a rotor shaft 130 extending through the central aperture 125, along the tubular housing 102 and through the central aperture 126. A pulley wheel 131 is connected to, and rotatable with the rotor shaft 130, as per photobioreactor 1. A power transfer column 136 surrounds the rotor shaft 130 and includes four conductive rods 137 extending parallel with the longitudinal axis L of the photobioreactor 100. The conductive rods 137 are configured to supply power to the lighting unit 104 from power source 106.

Annular seals 127 provide a fluid-tight seal between each of the apertures 125, 126 and the power transfer column 136.

In this example, the rotor shaft 130 carries a plurality of radially extending plate members 132 that are spaced from one another and configured to rotate with the rotor shaft 130, in use. Unlike photobioreactor 1, the plate members 132 need not be light-transmissible. Each of the plate members 132 has first and second planar surfaces 132a, 132b. As shown more clearly in FIG. 8, the plate members 132 partition the internal volume into respective fluidly connected volumes V. Each volume V is bounded by adjacent plate members 132 and the side wall 120 of the housing 102.

The lighting unit 104 is configured to provide light to the internal volume of the photobioreactor 100 and includes a plurality lighting elements 140 arranged on each of the planar surfaces 132a, 132b of the plate members 132. The lighting elements 140 are provided as an LED array in this example. The lighting unit 104 further includes a contact element 141 on each of the radially extending plate members 132 to provide electrical contact between the lighting elements 140 and conductive rods 137. The plate members 132 are configured to emit light from their respective planar surfaces 132a, 132b in order to illuminate the internal volume. In use, each respective volume V receives light from lighting elements 140 of the planar surfaces 132a, 132b of adjacent plate members 132. The power source 106 includes a plurality of carbon brushes 160 in contact with a commutator 161. The conductive rods 137 are, in turn, in contact with the commutator 161 such that power can be supplied to the conductive rods 137 while the rotor shaft 130 is rotated.

In use, current is supplied to the carbon brushes 160, which is then supplied to each of the conductive rods 137 via the commutator 161. This current then passes to the lighting elements 140 via the contact elements 141 such that the lighting elements 140 can be illuminated.

In the present example, the fluid contact elements 5 are as described above in relation to photobioreactor 1, and for brevity, will not be described further.

Further, the photobioreactor 100 is operated, in use, in a similar manner to photobioreactor 1. The principle difference is in the provision of light to the internal volume. In the present example, the lighting elements 140 are activated such that light is emitted into the internal volume from the planar surfaces 132a, 132b of the plate members 132.

Referring now to FIGS. 9 and 10, there is shown a fluid contact element 5 as described above in respect of the photobioreactor 1 of FIGS. 1 to 5. The fluid contact element 5 is arranged to be carried by a radially extending plate member 32, 132 (FIGS. 1 to 8) and mounted between adjacent radially extending plate members 32, 132. The fluid contact element 5 is in the form of a scoop or bucket and configured to collect liquid as the radially extending plate member 32, 132 is rotated.

The fluid contact element 5 has a pair of planar side walls 50, 51 connecting curved radially inner wall 53 and radially outer wall 52 that define bounding walls. The side walls 50, 51 and bounding walls 52, 53 define a scoop portion 54 for receipt of fluid during rotation of the plate member. An opening 55 is defined at a first end and a first end wall 56 is provided at a second end. The radially outer wall 52 is curved between the inlet 55 and rear wall 56. The radially inner wall 53 has a lip portion 53a and a curved portion 53b. The lip portion 53a extends from the inlet 55 to the curved portion 53b and the curved portion extends from the lip portion 53a to the first end wall 56. The lip portion 53a has the effect of narrowing the inlet 55 to the scoop portion 54.

A partition wall 57 is provided interstitially of the side walls 50, 51 and extends from the radially outer wall 52 to the radially inner wall 53. The partition wall 57 separates the scoop portion 54 into two separate volumes 54a, 54b.

The side walls 50, 51 are provided with apertures 58 configured to receive means for fixing the fluid contact elements 5 between radially extending plate members, in use. The lip portion 53a of the radially inner wall 53 has a plurality of perforations 59 extending therethrough in order to provide fluid communication between a location external of the fluid contact element 5 and the scoop portion 54. Further perforations 59 extend through each of the side walls 50, 51 proximate the lip portion 53a.

Referring now to FIGS. 11 and 12, there is shown a fluid contact element 105 according to an embodiment of the invention. The fluid contact element 105 is similar to fluid contact element 5 and like features are denoted by like references incremented by ‘100’. The fluid contact element 105 may be used in either of the photobioreactors 1, 100 described above. The fluid contact element 105 is arranged to be carried by a radially extending plate member 32, 132 (FIGS. 1 to 8) and mounted between adjacent plate members. The fluid contact element 5 is in the form of a scoop or bucket and configured to collect liquid as the plate member is rotated.

The fluid contact element 105 differs from fluid contact element 5 in that it does not include side walls. The side walls are provided by adjacent radially extending plate members, in use. The fluid contact element 105 includes radially outer wall 152 and curved radially inner wall 153 defining bounding walls that are interconnected by a first end wall 156 provided at a second end. In the present example, the first and second bounding walls 152, 153 and first end wall 156 are integrally formed.

In use, adjacent plate members, bounding walls 152, 153 and first end wall 156 define a scoop portion 154 for receipt of fluid during rotation of the plate member.

An inlet 155 is defined at a first end between the bounding walls 152, 153. The radially outer wall 152 is curved between the opening 155 and first end wall 156. The radially inner wall 153 has an upturned lip portion 153a and a curved portion 153b. The upturned lip portion 153a extends from the opening 155 to the curved portion 153b and the curved portion extends from the lip portion 153a to the first end wall 156. The upturned lip portion 153a has the effect of narrowing the opening 155 to the scoop portion 154.

The lip portion 153a has a plurality of perforations 159 extending therethrough providing fluid communication between a location external of the fluid contact element 105 and the scoop portion 154. The perforations 159 are provided in a row, extending across the width of the lip portion 153a in this example.

Referring now to FIGS. 13 and 14, there is shown a fluid contact element 205 according to an embodiment of the invention. The fluid contact element 205 is similar to fluid contact element 105 and like features are denoted by like references incremented by ‘100’.

The fluid contact element 205 may be used in either of the photobioreactors 1, 100 described above.

Fluid contact element 205 differs from fluid contact element 105 in that the curved radially inner wall 253 has an front retaining portion 253c extending from a curved portion 253b. The front retaining portion 253c extends towards the radially outer wall 252 and is configured to retain fluid within the scoop portion 254, in use. The inlet 255 is defined between the radially outer wall 252 and the front retaining portion 253c.

The radially inner wall 253 further includes a lip portion 253a that extends from the front retaining portion 253c towards the first end wall 256. In the present example, the lip portion 253a includes a plurality of perforations 259 extending therethrough providing fluid communication between a location external of the fluid contact element 205 and the trough portion 254. The perforations 259 are provided in a row, extending across the width of the lip portion 253a in this example.

In the present example, the radially outer and radially inner walls 252, 253 and first end wall 256 are integrally formed.

Referring now to FIGS. 15 and 16, there is shown a cross-section of a photobioreactor or bioreactor (hereinafter photobioreactor) 1, 100 in an in-use condition and with two different fluid phase arrangements. In the case of FIG. 15, the photobioreactor 1, 100 contains two phases, a liquid phase A and a gas phase B. In the case of FIG. 16, the photobioreactor 1, 100 contains solvent A and liquid phase B.

In FIG. 15, the volume of liquid A is significantly greater than the volume of gas B, resulting in a gas pocket at the top of the photobioreactor 1, 100. In use, the plate member 32, 132 rotates anti-clockwise causing the fluid contact elements 5, 105 to rotate around the internal circumference of the photobioreactor 1, 100. The fluid contact elements 5, 105 collect liquid A at the bottom of the cycle (i.e. proximate a six o'clock position in reference to FIG. 15) which is then transported around the internal circumference and emptied from the fluid contact elements 5, 105 when they are located at the top of the cycle (i.e. proximate a twelve o'clock position in reference to FIG. 15).

When the fluid contact elements 5, 105 are at the top of the cycle, they are located within the gas pocket. Gas B is collected by the fluid contact elements 5, 105 which is then transported below the surface of the liquid phase A as the fluid contact elements 5, 105 continue to be rotated. The apertures or perforations in the fluid contact elements 5, 105 (described above in FIGS. 9 to 14) results in the gas B being released whilst the fluid contact elements 5, 105 are submerged. This causes gas bubbles C to rise through the liquid phase A towards the gas pocket.

In FIG. 16, the volume of liquid B is significantly greater than the volume of solvent A. Due to the difference in density of the solvent A and liquid B, a pool of solvent B is formed at the bottom of the photobioreactor 1, 100. As described above in relation to FIG. 15, in use, the plate member 32, 132 rotates anti-clockwise causing the fluid contact elements 5, 105 to rotate around the internal circumference of the photobioreactor 1, 100. The fluid contact elements 5, 105 collect solvent A at the bottom of the cycle (i.e. proximate a six o'clock position in reference to FIG. 16) which is then transported around the internal circumference through the liquid B. In this case, the apertures or perforations in the fluid contact elements 5, 105 (described above in FIGS. 9 to 14) create a rain effect due to releasing a portion of the solvent A at a given orientation in the form of droplets D. The droplets D fall through the liquid B towards the solvent A. The rain effect increases the surface area of the solvent A in contact with liquid B and also encourages mixing of the waste liquid stream B and the solvent A. The rain effect also provides a cleaning effect to the plate members 32, 132 helping to reduce the build-up of algae or biofilm and therefore maintain the light emissive properties thereof in the event that the bioreactor is operated as a photobioreactor.

Referring now to FIG. 17, there is shown a cross-section of a photobioreactor or bioreactor (hereinafter photobioreactor) 1, 100 in an in-use condition and with the fluid phase arrangement of FIG. 15 and fluid contact element 5, 105. The arrangement is as described above in respect of FIG. 15 and for the sake of brevity will not be described in detail. However, it is shown that whilst the fluid contact elements 5, 105 are rotated anti-clockwise, the released gas bubbles C swirl in a clockwise direction as depicted by arrows E. Furthermore, when using fluid contact elements 5, 105, the majority of gas bubbles C are released within segment F.

Referring now to FIG. 18, there is shown a cross-section of a photobioreactor or bioreactor (hereinafter photobioreactor) 1, 100 in an in-use condition and with the fluid phase arrangement of FIG. 15 and fluid contact element 205. As described above, fluid contact element 205 differs from fluid contact elements 5, 105 in that it includes a has an front retaining portion (253c, FIG. 13). The phase arrangement is as described above in respect of FIG. 15 and for the sake of brevity will not be described in detail.

However, it is shown that whilst the fluid contact elements 205 are rotated anti-clockwise, the released gas bubbles C also swirl in an anti-clockwise direction as depicted by arrows E. Furthermore, when using fluid contact elements 205, the majority of gas bubbles C are released within segment G which is different than segment F and further on in the cycle.

Referring now to FIG. 19, there is shown a concentric scoop assembly 300 for use in a bioreactor 1, 100 as described above in respect of FIGS. 1 and 6. More specifically, the concentric scoop assembly 300 is configured to be received with the tubular housing 2, 102 of those embodiments.

The concentric scoop assembly 300 has a longitudinal axis L′ configured to correspond with the longitudinal axis L of the bioreactor 1, 100, in use. The concentric scoop assembly 300 comprises a plurality of circumferentially spaced fluid contact elements 305. The fluid contact elements 305 are defined by an inner tube 310 and an outer tube 312, wherein the inner and outer tubes 310, 312 are concentric and define an annulus H.

A plurality of annular walls 314 are provided within the annulus H and are equally spaced along the longitudinal axis L. The annular walls 314 connect the inner and outer tubes 310, 312. Furthermore, a plurality of longitudinally extending walls 316 are equally spaced around the annulus H and also connect the inner and outer tubes 310, 312.

Each of the plurality of scoops 305 is defined by a portion of the inner and outer tubes 310, 312, a pair of adjacent annular walls 314 and a pair of adjacent longitudinally extending walls 316. More specifically, the fluid contact elements 305 are provided as scoops/buckets in this example arranged in columns and have a pair of side walls 350, 351 defined by adjacent annular end walls 314. A curved radially inner wall 353 is provided by the inner tube 310 and a curved radially outer wall 352 is provided by the outer tube 312. First and second end walls 356a, 356b are provided by adjacent longitudinally extending walls 316. The walls define a scoop/bucket portion 354 for receipt of fluid during rotation of the concentric scoop assembly 300.

The inner tube 310 has a plurality of perforations or apertures 359 extending therethrough, arranged in longitudinally extending lines 359a. A plurality of such lines 359a is provided equally spaced around the inner tube 310, such that each line provides fluid communication between the internal volume of the bioreactor and each scoop/bucket portion 354 of a respective column.

The outer tube 312 also has a plurality of perforations or apertures 355, in the form of slots in this embodiment, extending therethrough and arranged in longitudinally extending lines 355a. A plurality of such lines 355a is provided equally spaced around the outer tube 312, such that each line provides fluid communication between an annular space defined between the tubular housing 2, 102 of the bioreactor 1, 100 and each scoop/bucket portion 354 of a respective column, in use.

It will also be appreciated by those skilled in the art that any number of combinations of the aforementioned features and/or those shown in the appended drawings provide clear advantages over the prior art and are therefore within the scope of the invention described herein.

1. A bioreactor comprising:
a housing defining an internal volume configured to hold a body of fluid;
at least one inlet for ingress of fluid into the internal volume;
at least one outlet for egress of fluid from the internal volume;
a rotor assembly comprising a rotatably mounted rotor shaft;
a plurality of fluid contact elements rotatable with the rotor shaft;
wherein at least one fluid contact element comprises a scoop having a curved radially inner wall, the curved radially inner wall being concave when viewed from a central axis of the housing;
wherein adjacent fluid contact elements are spaced apart in a circumferential direction.

2. A bioreactor according to claim 1, wherein the radially inner wall comprises one or more apertures.

3. A bioreactor according to claim 2, wherein the radially inner wall comprises a lip portion, the one or more apertures being formed in the lip portion.

4. A bioreactor according to claim 1, wherein the at least one fluid contact element further comprises:
a radially outer wall,
side walls extending between the radially inner and outer walls; and
an opening between the radially inner and outer walls at one end of the side walls.

5. A bioreactor according to claim 4, wherein at least one fluid contact element further comprises a first end wall extending between the radially inner and outer walls at the other end of the side walls.

6. A bioreactor according to claim 4, wherein one or each of the side walls comprises one or more apertures.

7. A bioreactor according to claim 4, wherein the radially inner wall comprises one or more apertures adjacent to the opening between the radially inner and outer walls.

8. A bioreactor according to claim 5, wherein the side walls together with the radially inner and outer walls and first end wall describe a receptacle for holding fluid.

9. A bioreactor according to claim 5, wherein the radially inner and outer walls and first end wall are integrally formed.

10. A bioreactor according to any claim 4, wherein the radially inner wall comprises a front retaining portion extending from a first end thereof towards the radially outer wall and configured to retain fluid within the liquid contact element, in use, wherein the opening is defined between the radially outer wall and front retaining portion.

11. A bioreactor according to claim 10, wherein the front retaining portion comprises a lip portion extending therefrom towards the first end wall of the fluid contact element.

12. A bioreactor according to claim 4, wherein the fluid contact element comprises a partition wall disposed between the side walls, wherein the partition wall separates the fluid contact element into separate volumes.

13. A bioreactor according to claim 1, wherein the or each fluid contact element is individually removably mounted within the housing.

14. A bioreactor according to claim 1, wherein the rotor shaft extends from an exterior of the housing into the internal volume.

15. A bioreactor according to claim 14, comprising at least one plate member mounted on, and configured to rotate with, the rotor shaft within the internal volume.

16. A bioreactor according to claim 15, comprising a plurality of spaced apart plate members mounted on the rotor shaft and a plurality of liquid contact elements extending between adjacent plate members.

17. A bioreactor as claimed in claim 1, wherein the bioreactor is a photobioreactor and comprises a source of electromagnetic radiation configured to transmit electromagnetic radiation to the internal volume.

18. A bioreactor according to claim 17, wherein the source of electromagnetic radiation is located externally of the housing and the bioreactor comprises transmission means for transmitting the electromagnetic radiation to the at least one plate member, wherein the plate member is configured to direct the electromagnetic radiation from a surface of the at least one plate member into the internal volume.

19. A bioreactor as claimed in claim 18, wherein the transmission means is rotatable with the rotor shaft.

20. A bioreactor as claimed in claim 19, wherein the rotor shaft comprises the transmission means.

21. A bioreactor according to claim 18, wherein the transmission means comprises a beam deflector arranged to direct electromagnetic radiation to the at least one plate member, in use, wherein the beam deflector comprises a surface etching or insert.

22. A bioreactor according to claim 15, wherein the at least one plate member is transmissive to the electromagnetic radiation.

23. A bioreactor according to claim 22, wherein the at least one plate member comprises at least one beam deflector which deflects electromagnetic radiation out of the plate member into the internal volume, in use.

24. A bioreactor as claimed in claim 18, wherein the means for projecting the electromagnetic radiation comprises at least one source of electromagnetic radiation mounted on a planar surface of the at least one plate member.

25. A bioreactor according to claim 1, wherein the rotor shaft and at least one plate member are submersible in the body of fluid.

26. A bioreactor according to claim 1, wherein, in use, the housing is arranged with its longitudinal axis substantially horizontally disposed.

27. A bioreactor comprising:
a housing defining an internal volume configured to hold a body of fluid;
at least one inlet for ingress of fluid into the internal volume;
at least one outlet for egress of fluid from the internal volume;
a rotor assembly comprising a rotatably mounted rotor shaft;
at least one fluid contact element rotatable with the rotor shaft;
wherein the at least one fluid contact element comprises a scoop having a wall with one or more apertures extending therethrough.

28. A bioreactor comprising:
a housing having an internal volume configured to hold a body of fluid;
at least one inlet for ingress of fluid into the internal volume;
at least one outlet for egress of fluid from the internal volume;
a rotor assembly comprising a rotatably mounted rotor shaft;
at least one fluid contact element rotatable with the rotor shaft;
wherein the at least one fluid contact element comprises a scoop.

29. A bioreactor according to claim 1, comprising one or more heating and/or cooling elements located either within or outside the internal volume.

30. A method of operating bioreactor according to claim 1, claim, wherein the bioreactor is more than 50% full of fluid, the method comprising rotating the rotor shaft such that at least one fluid contact element passes through the fluid.

31. A method according to claim 30, wherein the bioreactor is more than 50% full of liquid.

32. A method according to claim 30, wherein the fluid comprises two or more liquids having different densities, and the at least one fluid contact element passes through each of the two or more liquids.

33. A method according to claim 30, wherein the fluid comprises two or more liquids having different densities containing entrained solids, and the at least one fluid contact element passes through each of the two or more liquids containing entrained solids.

34. A method according to claim 30, wherein the fluid comprises a gas and a liquid, and the at least one fluid contact element passes through each of the gas and liquid.

35. A method according to claim 30, wherein the fluid comprises a gas and two or more liquids having different densities, and the at least one fluid contact element passes through each of the gas and two or more liquids.

36. A method according to claim 30, wherein the two or more liquids have different densities.

37. A method according to claim 30, wherein the fluid comprises a gas and two or more liquids and entrained solids within at least one of the liquids.

38. A method according to claim 36, wherein the at least one fluid contact element passes through each of the gas and two or more liquids with entrained solids.
